(19) United States
(12) Patent Application Publication
(10) Pub. No.: US 2020/0139154 A1
(43) Pub. Date: May 7, 2020

(54) MASK WITH MULTI-PROFILE FRAME AND CONNECTIBLE PROFILES

(71) Applicant: Medtec, Inc., Orange City, IA (US)

(72) Inventors: Roger F. WILSON, Sarasota, FL (US); Seth A. HULST, Orange City, IA (US); John STEFFEN, North Liberty, IA (US)

(21) Appl. No.: 16/495,990

(22) PCT Filed: Mar. 29, 2018
(86) PCT No.: PCT/US2018/025118

§ 371 Date: Sep. 20, 2019

Related U.S. Application Data
(60) Provisional application No. 62/479,608, filed on Mar. 31, 2017.
(60) Provisional application No. 62/541,881, filed on Aug. 7, 2017.

(51) Int. Cl. A61N 5/10

(52) U.S. Cl. CPC A61N 5/10; A61N 2005/1097

(57) ABSTRACT

A medical treatment or testing mask includes a first profile having a first fastening mechanism, and a second profile, that is separate from the first profile, having a second fastening mechanism that is fastenable to the first fastening mechanism. The mask further includes a first sheet of thermoplastic material attached to the first profile and to the second profile, wherein, when the second fastening mechanism is fastened to the first fastening mechanism, the first profile and the second profile create a single frame structure.

### REFERENCE TO RELATED APPLICATIONS

This application claims priority under 35 U.S.C. § 119, based on U.S. Provisional Patent Application No. 62/479,608 filed Mar. 31, 2017 and U.S. Provisional Patent Application No. 62/541,881 filed Aug. 7, 2017, the disclosures of which are hereby incorporated by reference herein.

### BACKGROUND

Certain types of medical treatments or tests require that a portion of a human body be held in a same position to facilitate performance of the medical treatment or test upon that portion of the body. For example, when brain cancer patients undergo radiation treatment, their heads must be maintained in a precise, repeatable location for the treatment such that the underlying position of the brain tumor is fixed in space for the duration of the radiation treatment or treatments. Various different techniques have been used in the field of radiation oncology for holding body parts in a fixed position.

### BRIEF DESCRIPTION OF THE DRAWINGS

FIG. 1 depicts a mask according to a first exemplary embodiment;

FIG. 2 illustrates an example of the docking of the mask of FIG. 1 to an underlying base in an implementation in which the mask is designed to fit over, and around, a patient's head (not shown) to fix the position of the head upon the base;

FIG. 3 depicts details of the multi-part mask frame of the mask of the embodiment of FIG. 1;

FIG. 4 depicts an example of the profiles of the mask of FIG. 1 being mounted to a base in an exemplary implementation in which the base fastening mechanisms include fastening pins;

FIGS. 5A and 5B depict a close-up view of a profile of the mask of FIG. 1 being mounted to a base in the exemplary implementation in which the base fastening mechanisms include fastening pins;

FIGS. 6, 7A, 7B, and 8 illustrate the attachment of the profiles of the mask of FIG. 1 to one another to create a single continuous mask frame when the mask is mounted to a base;

FIGS. 9A and 9B include a pictorial presentation of an exemplary process for mounting the mask of FIG. 1 to a base to fix the position of a patient's head;

FIG. 10A illustrates an additional embodiment in which a mask includes the structure of the mask of FIG. 1, but the sheet of thermoplastic material of the mask further includes an extended area designed to stretch over shoulders of a patient;

FIG. 10B depicts a further embodiment that includes a dual mask structure having at least two separate sheets of thermoplastic material;

FIG. 11 is a pictorial depiction of the mask of FIG. 10 stretched over a patient and mounted to an underlying base;

FIGS. 12A and 12B depict additional exemplary implementations of the multi-profile mask of FIG. 1 in which the mask includes vertical brackets for attaching to the sheet of thermoplastic material;

FIGS. 13A, 13B, and 13C depict another exemplary implementation of the multi-profile mask of FIG. 1 in which the mask includes five profiles; and

FIGS. 14A and 14B depict a variation of the implementation of FIG. 13C in which angled brackets have been added to some the profiles for attaching to the sheet of thermoplastic material.

### DETAILED DESCRIPTION OF THE PREFERRED EMBODIMENTS

The following detailed description refers to the accompanying drawings. The same reference numbers in different drawings may identify the same or similar elements. The following detailed description does not limit the invention.

A technique, in the medical treatment/testing fields, for holding body parts in a fixed position uses heat-formable structures that include a sheet of retention material that is stretched over the body part of the patient. For example, for performing radiation treatment of a brain tumor, the heat-formable structure includes a mask having a sheet of retention material that is stretched over the patient's face. To form the mask over the patient's face, a hot water bath or oven may be first used to heat the material of the heat-formable structure such that the sheet of material becomes pliable and deformable. The heat-formable mask is then stretched over the patient's face, and the mask is allowed to cool and harden, permanently forming the mask to the shape of the face of the patient. As an example, a mask having a sheet of thermoplastic retention material, after heating, may be stretched over a patient's face, and then allowed to cool. Upon cooling, the mask, formed to the patient's face, creates a structure that can be used to hold the patient's head in a fixed position during radiation treatments. After the sheet of thermoplastic retention material of the mask is stretched over the body part of the patient, a frame portion of the mask is attached to a patient support table, a base, or other structure, using an attachment mechanism(s).

Exemplary embodiments described herein relate to a treatment/testing mask where the frame of the mask is divided into multiple “profiles,” with each profile having fastening mechanisms for attaching to the underlying patient support table, base, or other structure. Additionally, each of the separate profiles of the frame of the mask has at least one fastening mechanism for fastening the profiles to one another so as to create a single, continuous mask frame when the profiles are fastened to one another. As described herein, the multiple profiles of the multi-part mask frame each connect to a sheet of thermoplastic material which can be stretched over a body part that is to be treated or tested. Use of multiple profiles for the mask frame of a treatment/testing mask allows for the use of a larger piece of thermoplastic material that further enables a drape style of fitting the mask to the patient's body part. The drape style of fitting the mask to the body part requires less stretch in the thermoplastic material and allows finer control for a better fit and improved conformity to the patient's body part.

In one implementation described herein, the profile fastening mechanisms of each profile of the mask frame include snap fit joints that permit a secure snap lock of one profile to another profile. This secure snap lock adds stability when the mask is docked to the underlying table, base, or other structure. The stability induced by the secure snap lock is especially helpful in an implementation in which one of the profiles (e.g., the top profile) is secured to the base with only one attachment point. Once the profiles of the mask have been fastened together using, for example, the snap fit joints, the connection between the multiple profiles of the mask may be maintained even when the mask is detached/unmounted from the table, base or other structure. This conjoined mask frame assists in simplifying reattachment and reposition of the body part for each subsequent treatment/test. The snap fit joints of an exemplary implementation allow for a certain amount of limited rotation of each profile about a vertical axis so that the mask can be spread apart slightly in order to clear the patient's body part during detachment and reattachment.

A “mask,” as referred to herein, includes any structure having a material (e.g., a thermoplastic material) that can be pulled over any body part(s) of a patient to form fit the material to the body part(s). In some embodiments, a “mask” enables the body part to be immobilized and held in a specific position using a fastening mechanism(s) that may, or may not, be a component of the mask. Thus, a “mask,” as used herein, does not refer solely to a structure for placement over a patient's face or head, but includes any type of structure for placement over any body part, or any portion of the body (that may include multiple body parts), of a patient (e.g., a structure that pulls over a pelvis of a patient).

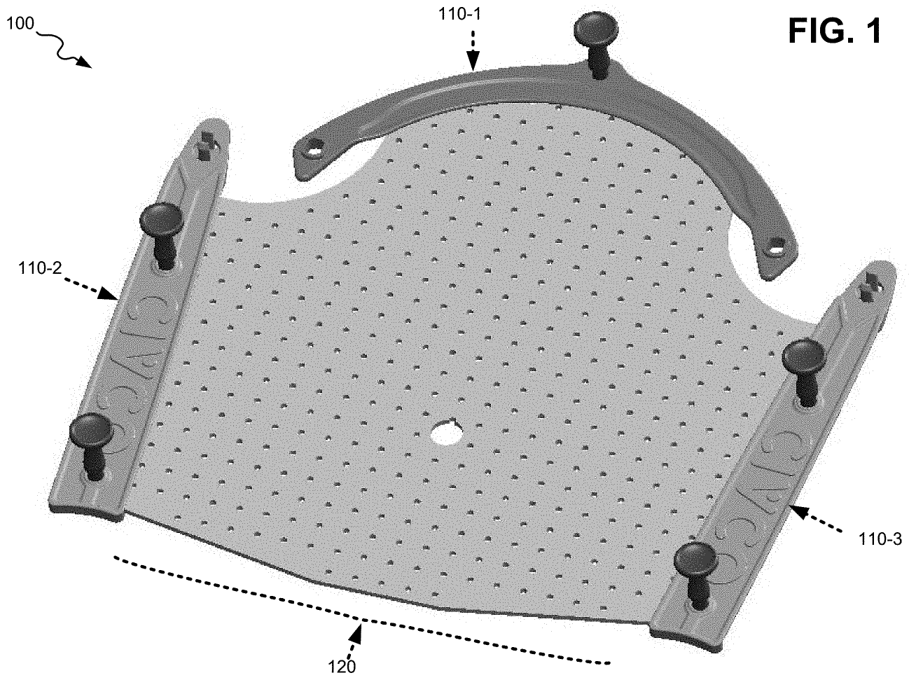

FIG. 1 depicts a mask 100 according to a first exemplary embodiment. As shown, mask 100 may include a multi-part mask frame that further includes multiple portions or profiles 110-1, 110-2 and 110-3 (generically referred to herein as a “profile 110” or “profiles 100”). Each profile 110 includes a separate portion of a frame to which a sheet of thermoplastic material 120 may be attached. As described further below, the multiple profiles 110-1, 110-2, and 110-3 may be fastened together, using fastening mechanisms at the time the mask 110 is attached to a base or other structure, to create a mask 100 having a single continuous mask frame structure. In the embodiment depicted in FIG. 1, the shape of the mask frame, when profiles 110-1, 110-2 and 110-3 are fastened together, may be a U-shape, where the sheet of thermoplastic material 120 is attached within the inner region of the “U.” Other shapes, however, may alternatively used, such as, for example, a rectangular frame having one open side, a square frame having one open side, a triangular frame having one open side, etc. The shape of the mask frame may be designed to fit over, or around, a specific body part, or portion of a body (e.g., that includes multiple body parts), of a patient. Each of the profiles 110 of the mask frame may be formed from various types of materials, including metal, plastic, carbon fiber, or a composite material.

The sheet of thermoplastic material 120 attached to each of the profiles 110 may include, as depicted in FIG. 1, a sheet of material formed in a mesh pattern including a number of holes, openings, or perforations. Thermoplastic material 120 may include material that is stretchable, for example, up to 400% or more from its original dimensions. The shape of the sheet of thermoplastic material 120 may be designed to fit over, or around, a specific body part, or portion of a body, of a patient. The sheet of thermoplastic material 120 may, in one embodiment, be made of polycaprolactone (PCL), or a PCL/polyurethane blend. Other types of thermoplastic material, including other types of polymers, may, however, be alternatively used. The sheet of thermoplastic material 120 may be affixed within the inner region of the multi-part mask frame, including profiles 110-1 through 110-3, using various techniques. For example, the sheet of thermoplastic material 120 may be glued to each of the profiles 110 of the mask frame. As a further example, the sheet of thermoplastic material 120 may be affixed to each of the profiles 110 of the mask frame using heat to melt material 120 such that it bonds with, or adheres to, the profiles 110. Generation of the heat to melt material 120 to bond with, or adhere to, the profiles 120 may include a direct application of heat (e.g., via a heated element, or a controlled flame), or an indirect application of heat (e.g., an ultrasonic welder that indirectly generates heat using ultrasonic waves). As another example, each profile 110 of the mask frame may include two separate, interlocking pieces that “snap” together. In this example, the edges of the sheet of thermoplastic material 120 are laid between the two interlocking pieces of the profile 110, and then the two pieces of profile 110 are “snapped” together, using fastening mechanisms, to hold the sheet of thermoplastic material 120 stretched tautly in the inner region of the profile 110.

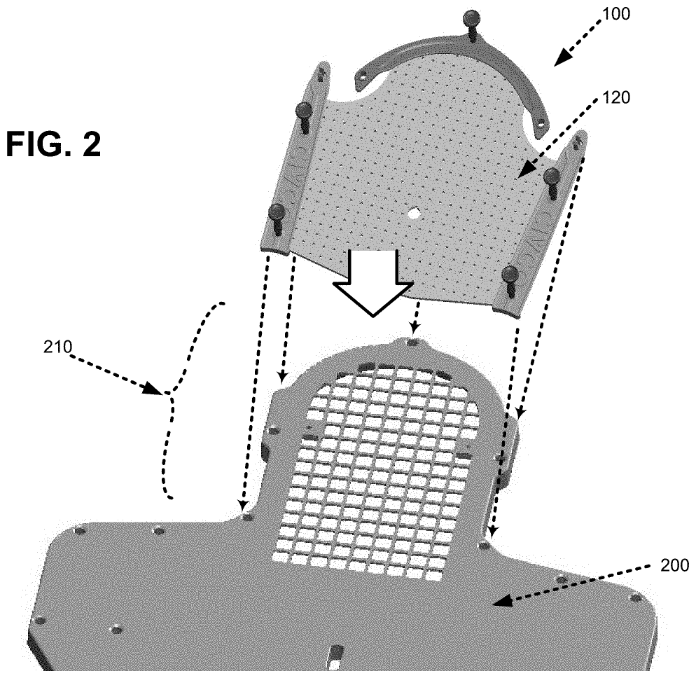

FIG. 2 illustrates an example of the docking of mask 100 to an underlying base 200 in an implementation in which mask 100 is designed to fit over, and around, a patient's head (not shown) to fix the position of the head upon base 200. Base 200 may, for example, be associated with a treatment table in a radiation therapy system. In the example of FIG. 2, once the sheet of thermoplastic material 120 has been properly prepared (e.g., immersed in a hot water bath having water heated to an appropriate temperature for heat forming thermoplastic material 120), mask 100 may be placed over the face of the patient (not shown), and then the sheet of thermoplastic material 120 is stretched downwards such that the material of mask 100 stretches to form fit to the patient face. Upon stretching the sheet of thermoplastic material 120 of mask 100 to reach the upper surface of a head region 210 of base 200, mask 100 may be attached to head region 210 of base 200, as described in further detail below.

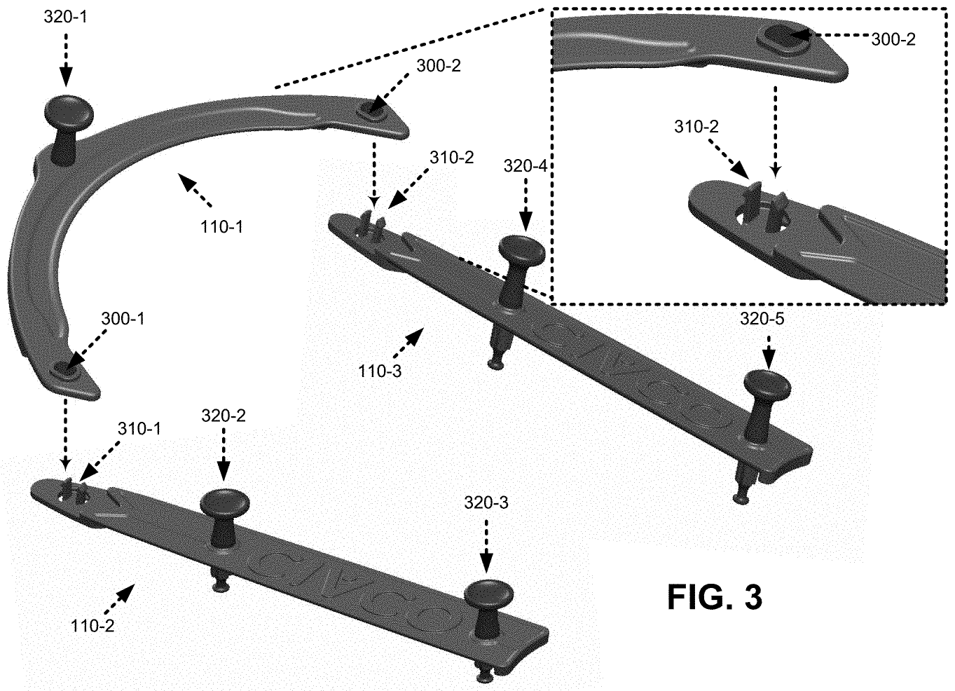

FIG. 3 depicts details of the multi-part mask frame of mask 100 of the embodiment of FIG. 1. As shown, profile 110-1 is formed in a semi-circular shape and includes profile fastening mechanisms 300-1 and 300-2, and base fastening mechanisms 320-1. Profile fastening mechanisms 300-1 and 300-2 are disposed at each end of the semi-circular shape of profile 110-1. Base fastening mechanisms 320-1, as shown, is disposed at a mid-point of the semi-circular shape of profile 110-1. In the implementation shown in FIG. 3, profile fastening mechanisms 300-1 and 300-2 include fastening holes configured to receive corresponding mating fastening mechanisms 310-1 and 310-2 from profiles 110-2 and 110-3, and base fastening mechanisms 320-1 includes a fastening pin that inserts through a pin hole in profile 110-1 into a corresponding pin hole in base 200 (not shown). The fastening pin may, for example, include an expansion type lock for attaching each profile 110 to base 200.

As further shown in FIG. 3, profile 110-1 and 110-3 each include a longitudinal shape, with profile 110-2 having a profile fastening mechanism 310-1 disposed at one end and profile 110-3 having a profile fastening mechanism 310-2 disposed at one end. Profile 110-2 further includes base fastening mechanisms 320-2 and 320-3, and profile 110-3 further includes base fastening mechanisms 320-4 and 320-5. In the implementation shown in FIG. 3, profile fastening mechanisms 310-1 and 310-2 each include two beam catches that act, in conjunction with the fastening holes of fastening mechanisms 300-1 and 300-2, as snap fit joints that serve to fasten profile 110-1 to profiles 110-2 and 110-3. Therefore, the fastening hole of fastening mechanism 310-1 acts in conjunction with the two beam catches of fastening mechanism 300-1 as a snap fit joint for fastening profile 110-1 to profile 110-2. The fastening hole of fastening mechanism 310-2 acts in conjunction with the two beam catches of fastening mechanism 300-2 as a snap fit joint for fastening profile 110-1 to profile 110-3. The use of snap fit joints, in the implementation depicted, enables attachment and detachment of the profiles 110 to be performed in a simple, user friendly, reliable manner. Additionally, in the implementation shown in FIG. 3, base fastening mechanisms 320-2 and 320-3 of profile 110-2, and base fastening mechanisms 320-4 and 320-5 of profile 110-3, each includes a fastening pin that inserts through a pin hole in the profile 110 into a corresponding pin hole in base 200 (not shown) for mounting the profiles 110 to base 200.

Though profile fastening mechanisms 300-1, 300-2, 310-1 and 310-2 have been described as being implemented as snap fit joints for fastening profile 110-1 to profiles 110-2 and 110-3, other types of profile fastening mechanisms may be alternatively used. Furthermore, though base fastening mechanisms 320-1 through 320-5 have been described as being implemented as fastening pins that insert through a pin hole in the profiles 110 into corresponding pin holes in base 200 (not shown), other types of base fastening mechanisms may be alternatively used.

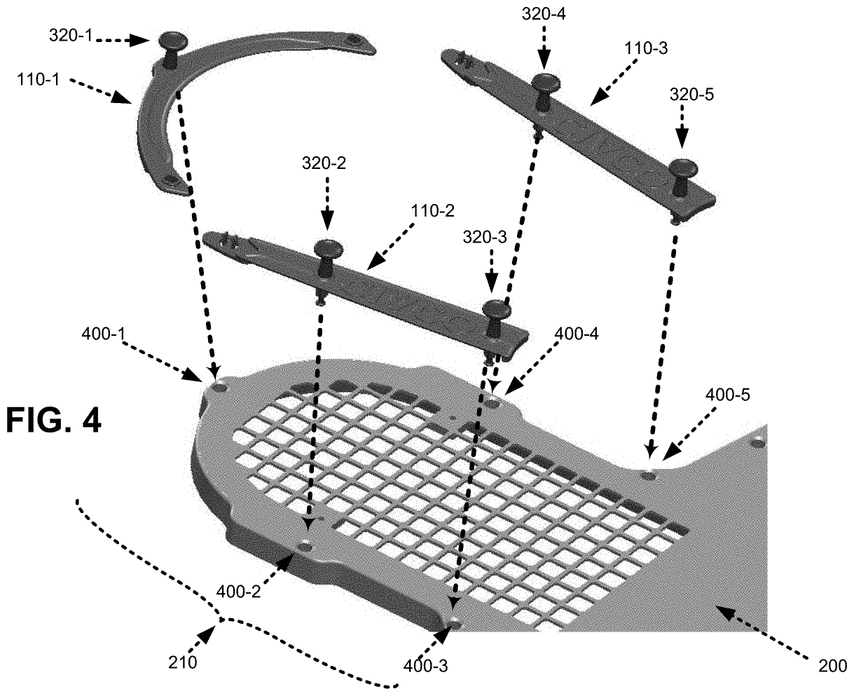

FIG. 4 depicts an example of the profiles 110 of mask 100 being mounted to head region 210 of base 200 in an exemplary implementation in which base fastening mechanisms 320-1 through 320-5 include fastening pins. As shown, when profile 110-2 is mounted to head region 210 of base 200, fastening pin 320-2 is inserted through a first mounting hole in profile 110-2 into a base attachment hole 400-2 in base 200, and fastening pin 320-3 is inserted through a second mounting hole in profile 110-2 into a base attachment hole 400-3 in base 200. As further shown, when profile 110-3 is mounted to head region 210 of base 200, fastening pin 320-4 is inserted through a first mounting hole in profile 110-3 into a base attachment hole 400-4 in base 200, and fastening pin 320-5 is inserted through a second mounting hole in profile 110-3 into a base attachment hole 400-5 in base 200. As additionally shown, when profile 110-1 is mounted to head region 210 of base 200 (including fastening to profiles 110-2 and 110-3 as described further below), fastening pin 320-1 is inserted through a mounting hole in profile 110-1 into a base attachment hole 400-1 in base 200.

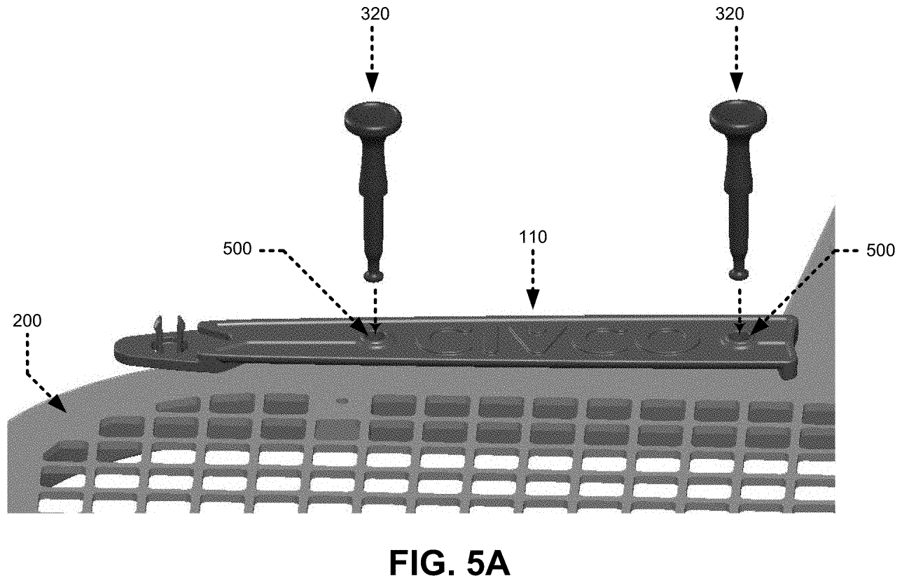

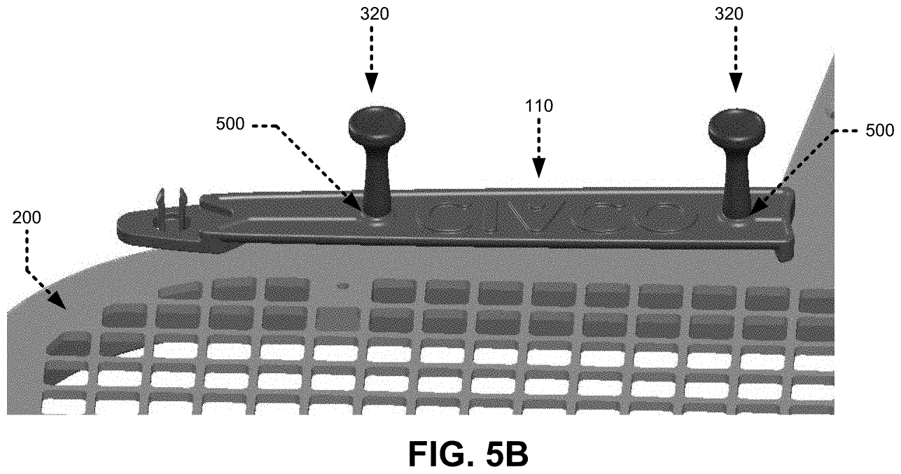

FIGS. 5A and 5B depict a close-up view of a profile 110 being mounted to base 200 in the exemplary implementation in which base the fastening mechanism 320 include fastening pins. Once profile 110 is placed in an appropriate position upon the upper surface of base 200, as shown in FIG. 5A, fastening pins 320 may be inserted through mounting holes 500 in profile 110. As further shown in FIG. 5B, once fastening pins 320 are inserted through the mounting holes 500 in profile 110, downward force may be applied until a lower portion of the fastening pins 320 fully extends into a corresponding base attachment hole 400 (not shown) in base 200, anchoring profile 110 in a mounted position upon base 200. This anchoring process may be repeated for each fastening pin 320 on each profile 110 mounted to base 200.

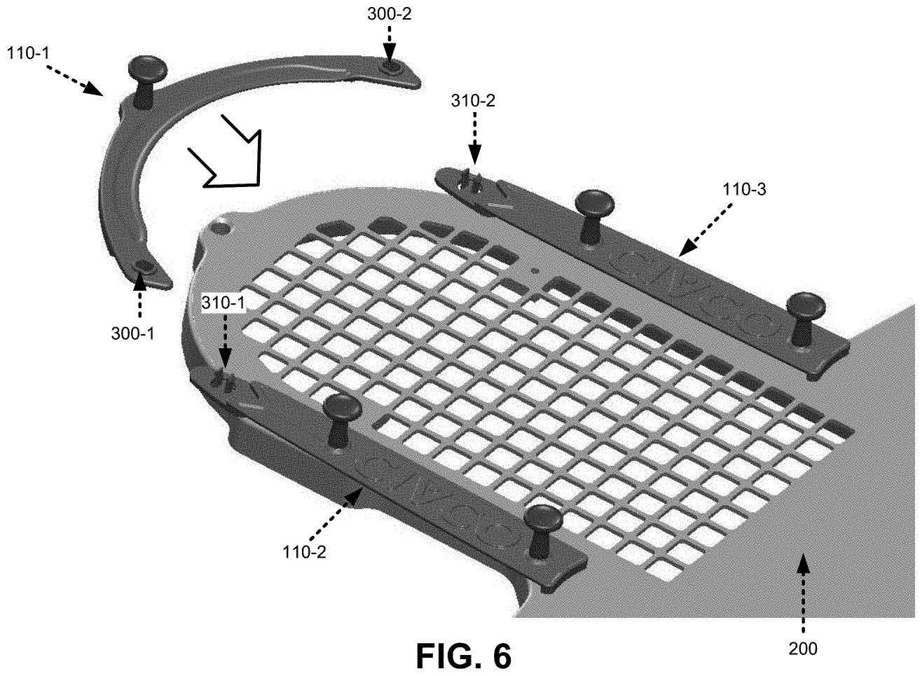

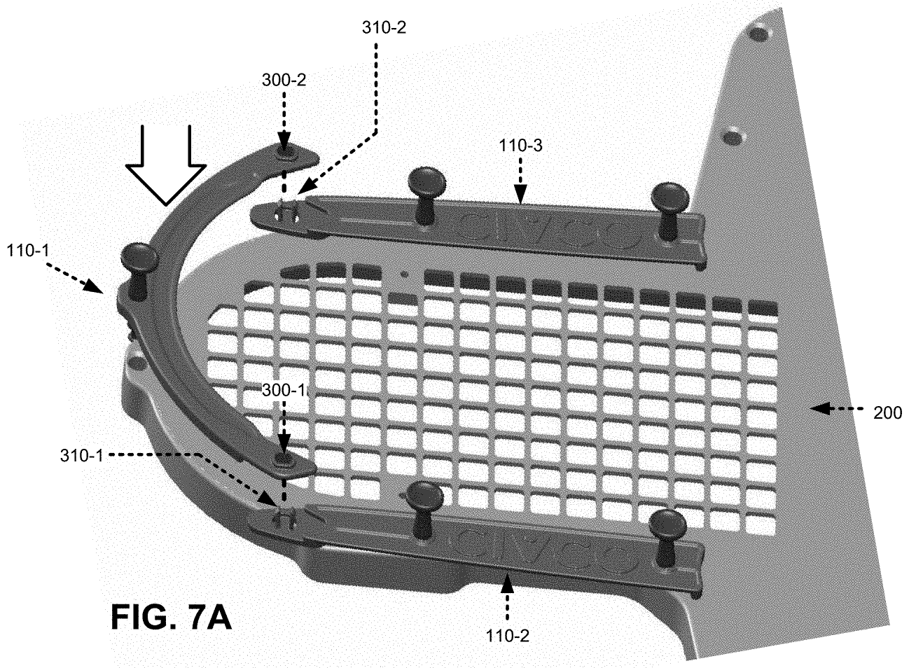

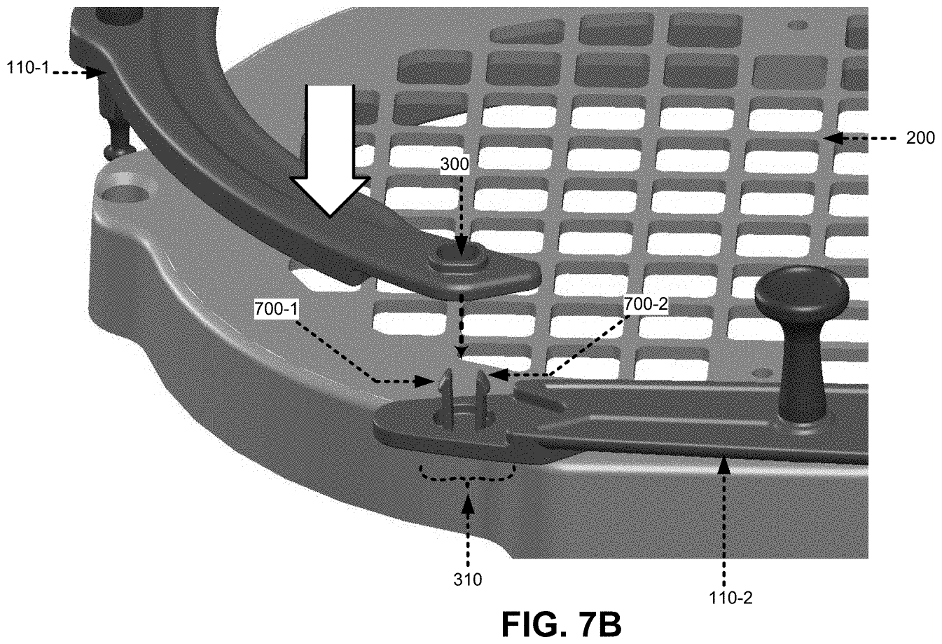

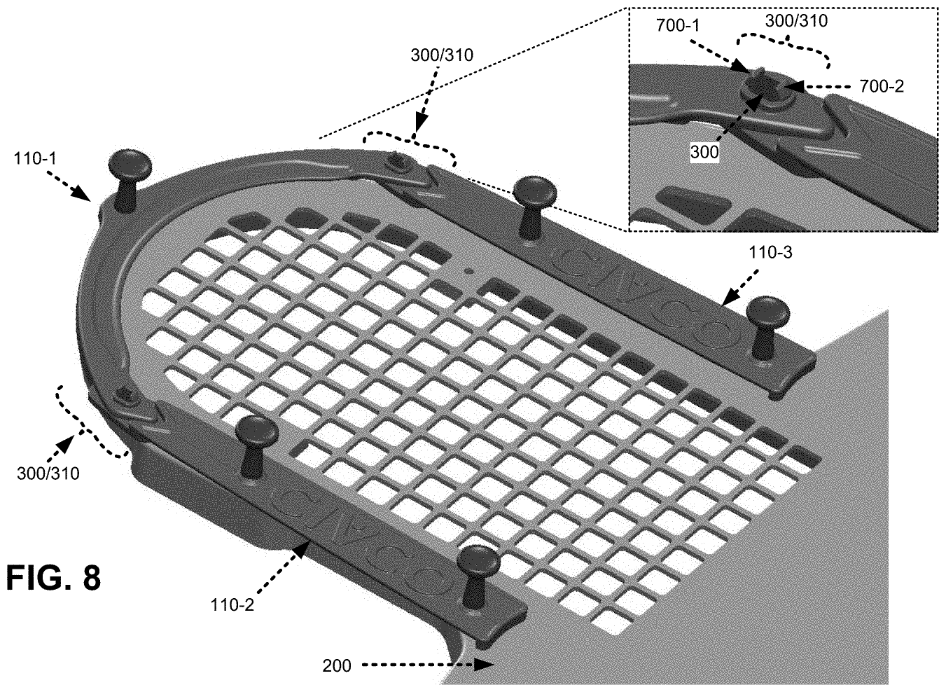

FIGS. 6, 7A, 7B, and 8 illustrate the attachment of profile 110-1 to profiles 110-2 and 110-3, to create a single continuous mask frame, when mask 100 is mounted to base 200. Once profiles 110-2 and 110-3 are mounted to base 200, as shown in FIG. 6, profile 110-1 may be moved downward, stretching the sheet of thermoplastic material 120 (not shown) over the body part, to mate fastening mechanism 300-1 of profile 110-1 with fastening mechanism 310-1 of profile 110-1, and to mate fastening mechanism 300-2 with fastening mechanism 310-2. As further shown in FIG. 7A, profile 110-1 is moved downwards until fastening mechanism 300-1 is directly aligned over fastening mechanism 310-1 of profile 110-2, and fastening mechanism 300-2 is directly aligned over fastening mechanism 310-2 of profile 110-3. Once fastening mechanisms 300 of profile 110-1 are aligned over the fastening mechanisms 310, profile 110-1 may be moved directly downwards, as shown in FIG. 7B to mate fastening mechanism 300 with fastening mechanism 310. In an exemplary implementation in which fastening mechanism 300 and fastening mechanism 310 include a snap fit joint, the profile fastening hole of fastening mechanism 300 of the snap fit joint is mated with male beam catches 700-1 and 700-2 of fastening mechanism 310. Downwards motion of profile 110-1 causes beam catches 700-1 and 700-2 to extend upwards through the profile fastening hole until the beam catches “snap” into place within the corresponding female profile fastening hole. FIG. 8 depicts profiles 110-1, 110-2 and 110-3 with mated fastening mechanisms 300/310 such that a single continuous mask frame is created subsequent to attaching profile 110-1 to profiles 110-2 and 110-3, and to base 200. As can be seen in the break out view of FIG. 8 (upper right corner of FIG. 8), mated fastening mechanisms 300/310 include the upper portion (i.e., the catch) of male beam catches 700-1 and 700-2 extended up through female profile fastening hole 300 such that the catch extends out of the profile fastening hole 300 to “grab” onto the upper surface of the edge of profile fastening hole 300. Detachment of mated fastening mechanisms 300/310 may include “pinching” male beach catches 700-1 and 700-2, causing the beams to pivot inwards within profile fastening hole 300, such that the catches release from the upper surface of the edge of the profile fastening hole 300, and the profile 110 may then be lifted upwards and away from the underlying profile to which it is attached.

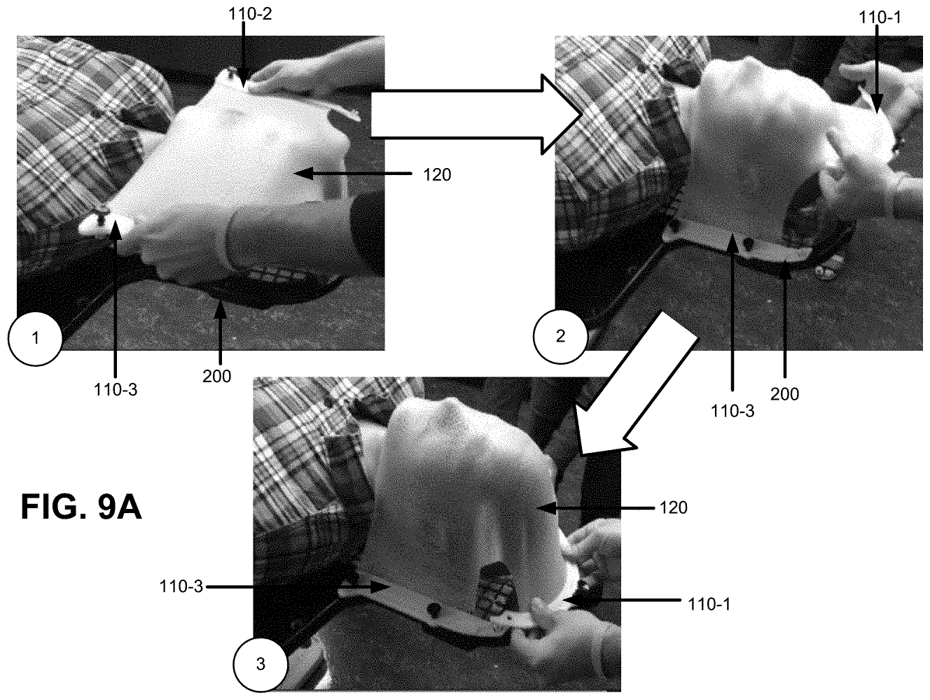

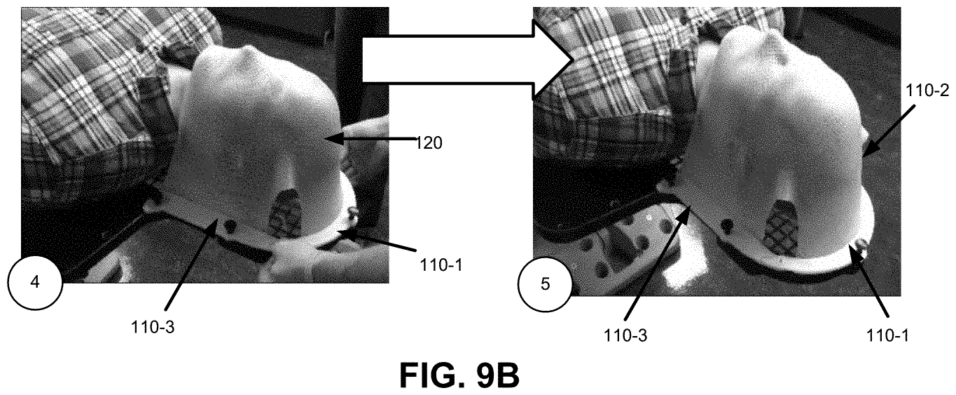

FIGS. 9A and 9B include a pictorial presentation of an exemplary process for mounting mask 100 to base 200 to fix the position of a patient's head, with the temporal sequence of mounting mask 100 to base 200 indicated by sequential integers (i.e., each disposed within a circle) associated with each image of the process. As shown at “1” in FIG. 9A, the sheet of thermoplastic material 120 of mask 100 is stretched over the face of the patient by holding onto profiles 110-2 and 110-3. As further shown at “2” in FIG. 9A, once profiles 110-2 and 110-3 are attached to base 200, profile 110-1 may be pulled downwards to stretch the remaining portion of the sheet of thermoplastic material 120 of mask 100 in the vicinity of the forehead and top of the head of the patient. Profile 110-1 is pulled downwards, as shown at “3” in FIG. 9A until the fastening mechanisms of profile 110-1 mate with the fastening mechanisms of profiles 110-2 and 110-3 (shown at “4” in FIG. 9B). At “5” in FIG. 9B, mask 100 is shown with profiles 110-1, 110-2, and 110-3 fastened to base 200, and profile 110-1 fastened to profiles 110-2 and 110-3 to create a single continuous mask frame.

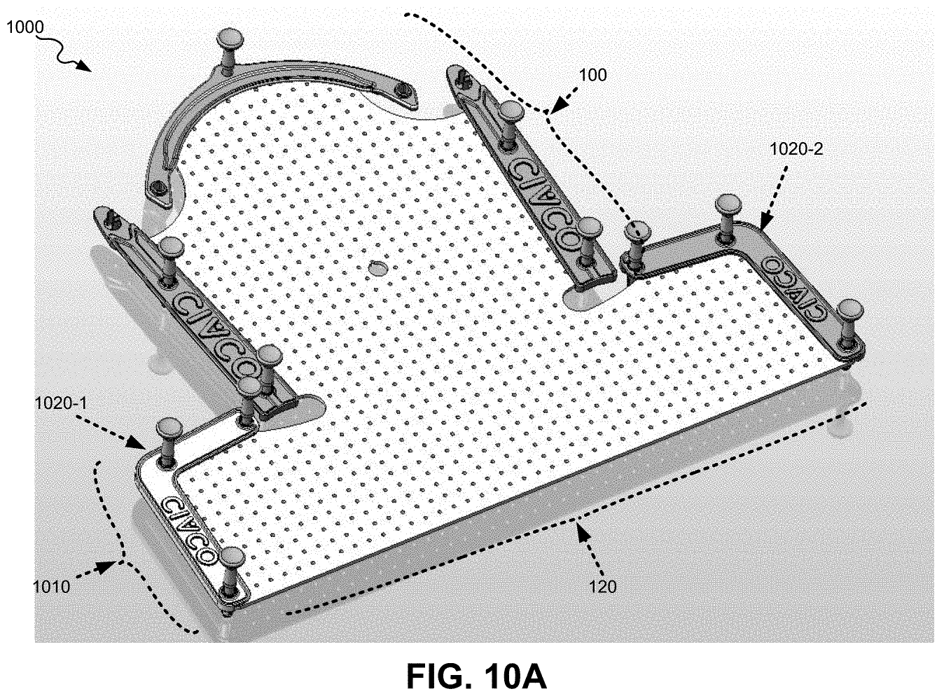

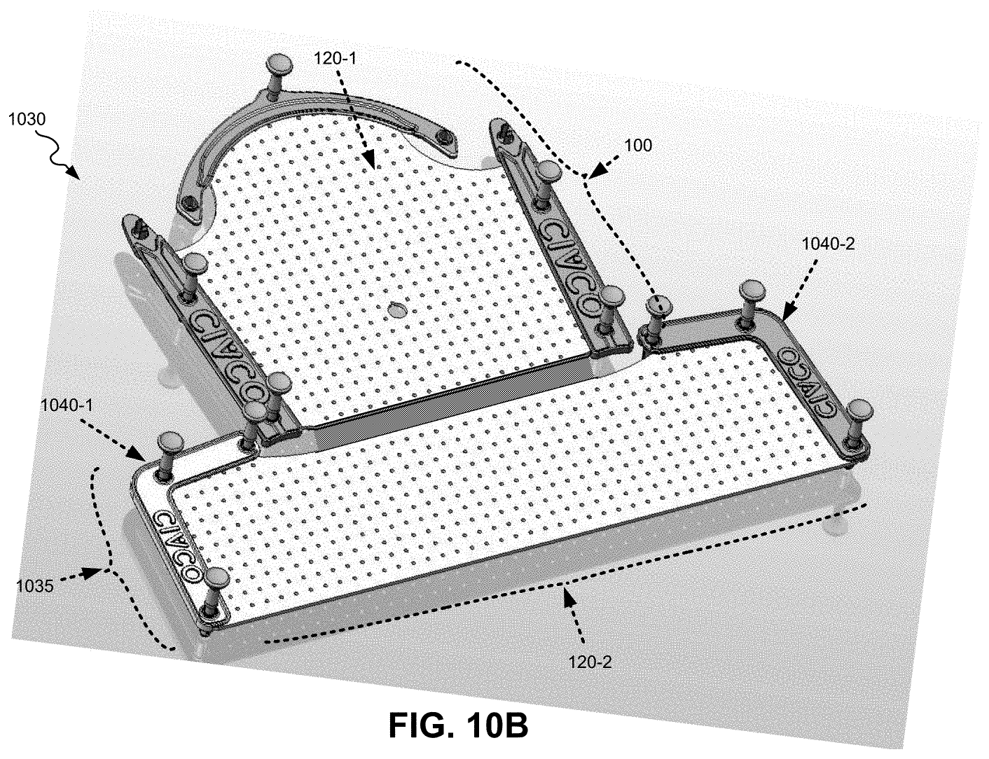

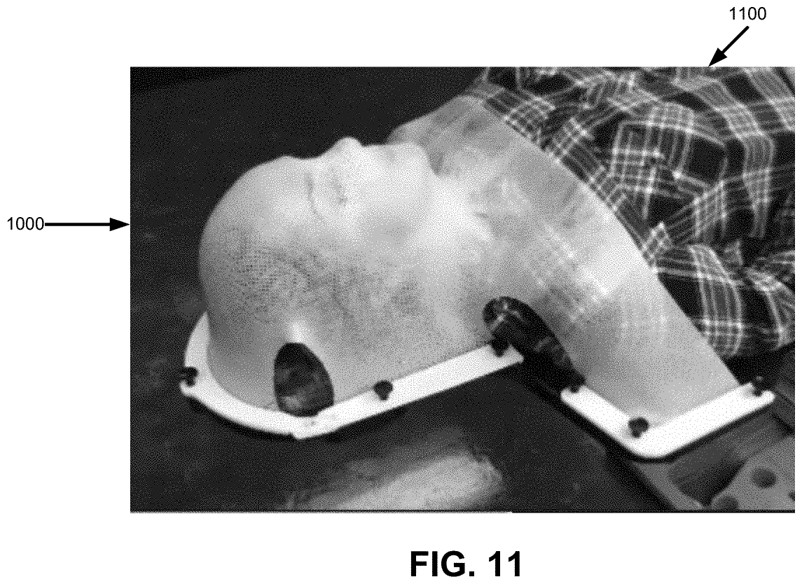

FIG. 10A illustrates an additional embodiment in which a mask 1000 includes the structure of mask 100 of FIG. 1, but the sheet of thermoplastic material 120 further includes an extended area 1010 of material designed to fit (e.g., stretch) over shoulders (i.e., a second body part) of a patient in addition to the patient's head (i.e., a first body part). The extended area 1010 of mask 1000 is attached to two additional profiles 1020-1 and 1020-2 that attach to the underlying base adjacent to the shoulders of the patient. In the embodiment of FIG. 10A, the two additional profiles 1020-1 and 1020-2 may attach to material 120 but not fasten to, or be part of, the single continuous frame created by profiles 110-1, 110-2, and 110-3 when these three profiles are fastened together. Profiles 1020-1 and 1020-2 are discontinuous portions of the frame structure that includes the continuous frame portion of profiles 110-1, 110-2, and 110-3. Therefore, in this embodiment, mask 100 may include multiple profiles (e.g., five) that connect to material 120 to create a discontinuous frame structure, where only a portion of the profiles (e.g., 3) connect together to form the continuous portion of the frame structure. The discontinuous frame structure, as shown in the example of FIG. 10, may include at least two profiles that attach to material 120 and which are fastenable to one another to create the single continuous frame portion, and may further include at least one additional profile that also attaches to material 120, but that is not fastenable to the continuous frame portion. As can be seen in the example of FIG. 10A, however, each profile of the continuous frame portion, and the discontinuous frame portion, of the mask 100 may be fastenable to an underlying base. FIG. 11 is a pictorial depiction of mask 1000 stretched over a patient 1100 and mounted to an underlying base.

FIG. 10B illustrates a further embodiment in which a dual mask 1030 includes the structure of mask 100 of FIG. 1, with a first sheet of thermoplastic material 120-1 connected between at least two connectible profiles (three connectible profiles depicted in the example of FIG. 10B) that attach to an underlying base structure (not shown). This embodiment also includes a second mask structure 1035 that includes a second sheet of thermoplastic material 120-2 connected between at least two profiles 1040-1 and 1040-2 that attach to the underlying base structure (not shown). The at least two profiles 1040-1 and 1040-2 may, or may not, be connectible to one another (shown as non-connectible in FIG. 10B). In this embodiment, the mask structure 100 is configured to fit over a first body part (e.g., a patient's head in the embodiment shown in FIG. 10B) or a first body portion, and the second mask structure 1035 is configured to fit over a second body part or a second, different body portion (e.g., a patient's upper torso, including shoulders). The exemplary embodiment of FIG. 10B, therefore, includes multiple, separate mask structures (two shown in the example of FIG. 10B), having multiple, separate, and unconnected sheets of thermoplastic material (e.g., 120-1 and 120-2), that fit over different body parts, different body portions, or different regions of a same body part or portion, but attach to an underlying base(s).

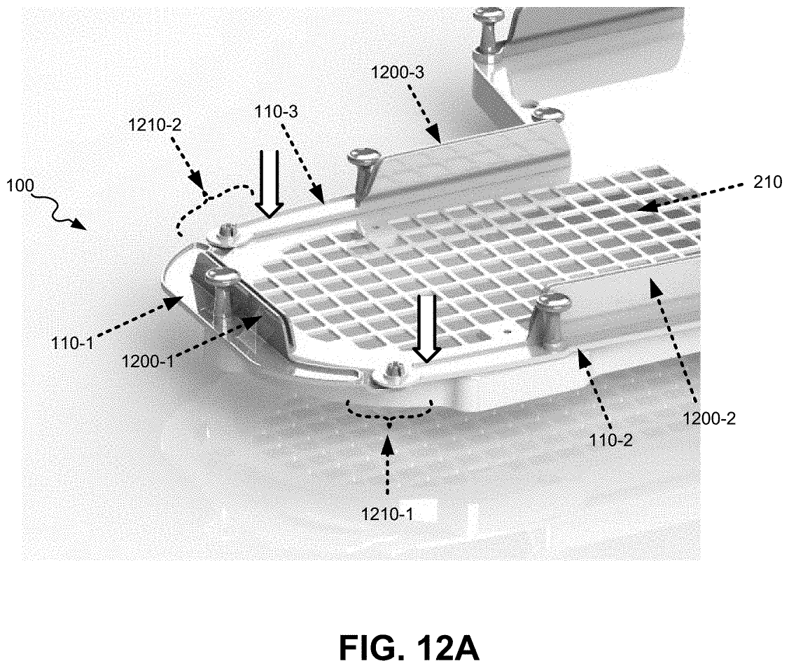

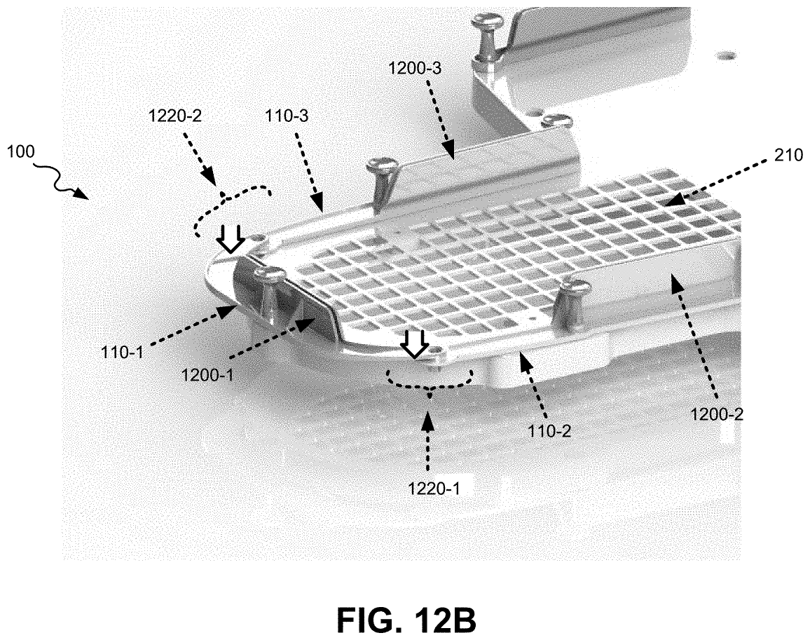

FIGS. 12A and 12B depict additional exemplary implementations of the multi-profile mask 100 of FIG. 1 in which mask 100 includes vertical brackets for attaching to the sheet 120 of thermoplastic material (not shown). As shown in FIG. 12A, profiles 110-1, 110-2, and 110-3 have a similar physical configuration to the implementation depicted in FIG. 1, with the addition of a first vertical bracket 1200-1 to profile 110-1, a second vertical bracket 1200-2 to profile 110-2, and a third vertical bracket 1200-3 to profile 110-3. Each vertical bracket 1200 may connect to an upper surface of a respective profile 110, and extend upwards perpendicularly to the upper surface of the profile. The appropriate edge of the sheet 120 of thermoplastic material (not shown in FIGS. 12A and 12B) may be inserted within each bracket 1200 and a bracket fastening mechanism (not shown) may be used to attach each edge of the sheet 120 to each bracket 1200. Various different types of bracket fastening mechanisms may be used that hold the edge of the sheet 120 within, and attach the edge to, a respective bracket 1200. FIG. 12A depicts an implementation in which fastening mechanisms 1210-1 and 1210-2 face upwards, and FIG. 12B depicts another implementation in which fastening mechanisms 1220-1 and 1220-2 face downwards. The fastening mechanisms 1210-1, 1210-2, 1220-1, and 1220-2 act as snap fit joints that serve to fasten profiles 110-2 and 110-3 to profile 110-1.

In the implementation of FIG. 12A, fastening mechanisms 1210-1 include a fastening hole disposed at the end of profile 110-2 that attaches to profile 110-1, and profile 110-1 includes a profile fastening mechanism that mates with the fastening hole. Additionally, fastening mechanisms 1210-2 include a fastening hole disposed at the end of profile 110-3 that attaches to profile 110-1, and profile 110-1 includes a profile fastening mechanism that mates with the fastening hole. In the depicted implementation, the profile fastening mechanism of fastening mechanisms 1210-1, disposed on profile 110-1, includes multiple beam catches, directed upwards perpendicular to base 210, that act in conjunction with the fastening hole of profile 110-2 as a snap fit joint for fastening profile 110-2 to profile 110-1. Furthermore, in the depicted implementation, the profile fastening mechanism of fastening mechanism 1210-2, disposed on profile 110-1, includes multiple beam catches, directed upwards perpendicular to base 210, that act in conjunction with the fastening hole of profile 110-3 as a snap fit joint for fastening profile 110-3 to profile 110-1. Therefore, once profile 110-1 has been attached to base 210, profile 110-2 may lowered onto to profile 110-1 to fasten them together by engaging the snap fit joint (i.e., fastening mechanisms 1210-1) that includes the multiple beam catches at a first end of profile 110-1 and the fastening hole disposed at the end of profile 110-2. Additionally, profile 110-3 may be lowered onto to profile 110-1 to fasten them together by engaging the snap fit joint (i.e., fastening mechanisms 1210-2) that includes the multiple beam catches at a second end of profile 110-1 and the fastening hole disposed at the end of profile 110-3.

In the implementation of FIG. 12B, fastening mechanisms 1220-1 include a fastening hole disposed at the end of profile 110-2 that attaches to profile 110-1, and profile 110-1 includes a profile fastening mechanism that mates with the fastening hole. Additionally, fastening mechanisms 1210-2 include a fastening hole disposed at the end of profile 110-3 that attaches to profile 110-1, and profile 110-1 includes a profile fastening mechanism that mates with the fastening hole. In the depicted implementation, the profile fastening mechanism of fastening mechanisms 1210-1, disposed on profile 110-1, includes multiple beam catches, directed downwards perpendicular to base 210, that act in conjunction with the fastening hole of profile 110-2 as a snap fit joint for fastening profile 110-2 to profile 110-1. Furthermore, in the depicted implementation, the profile fastening mechanism of fastening mechanism 1210-2, disposed on profile 110-1, includes multiple beam catches, directed downwards perpendicular to base 210, that act in conjunction with the fastening hole of profile 110-3 as a snap fit joint for fastening profile 110-3 to profile 110-1. Therefore, once profiles 110-2 and 110-3 have been attached to base 210, profile 110-1 may be lowered onto both profiles 110-2 and 110-3 to fasten profile 110-1 to profiles 110-2 and 110-3, by engaging the snap fit joints (i.e., fastening mechanisms 1220-1 and 1220-2) that includes the multiple beam catches at a first end of profile 110-1 and the fastening hole disposed at the end of profile 110-2, and the multiple beam catches at a second end of profile 110-1 and the fastening hole disposed at the end of profile 110-3.

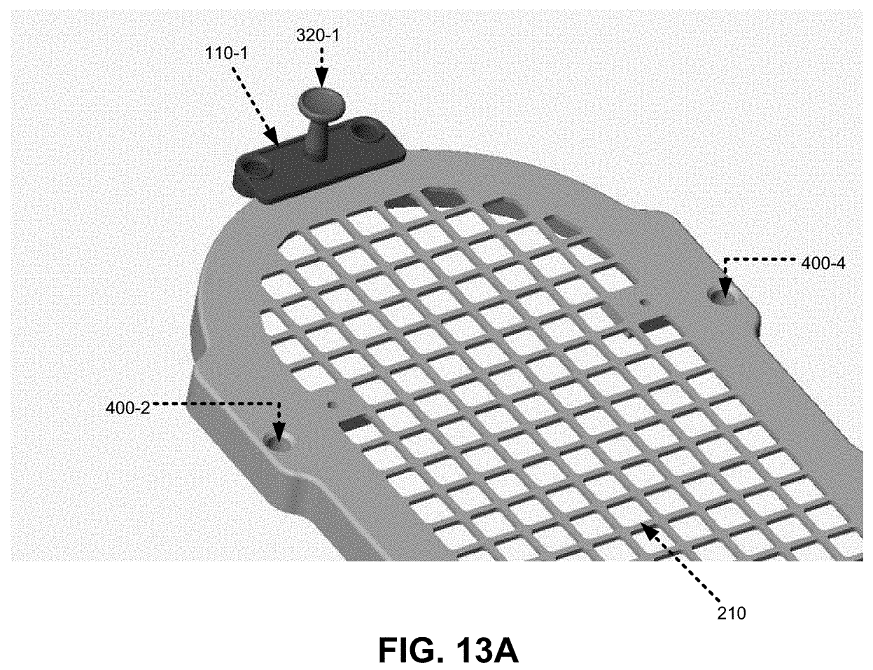

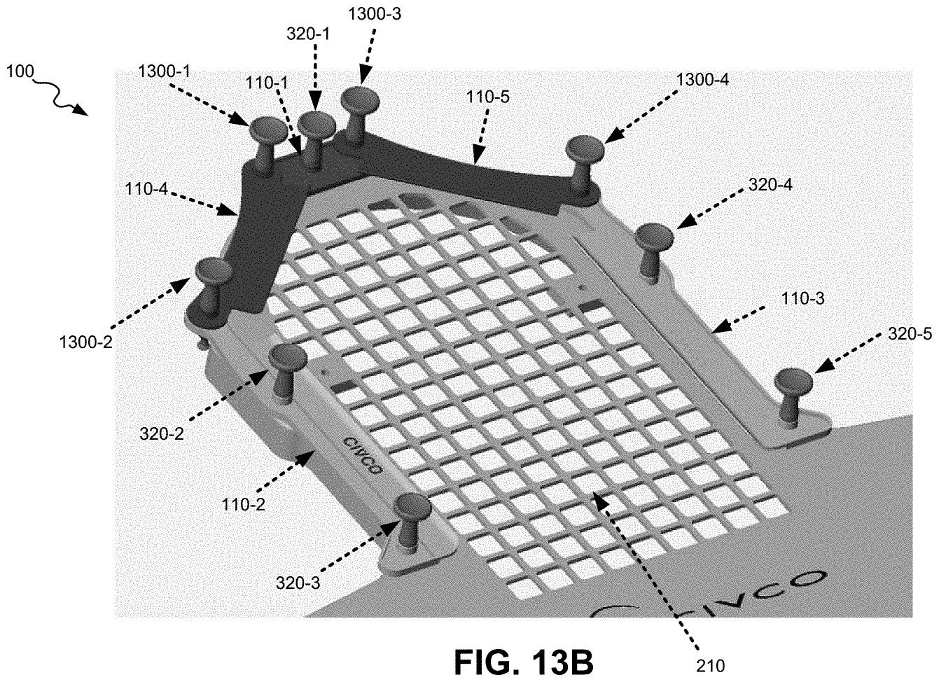

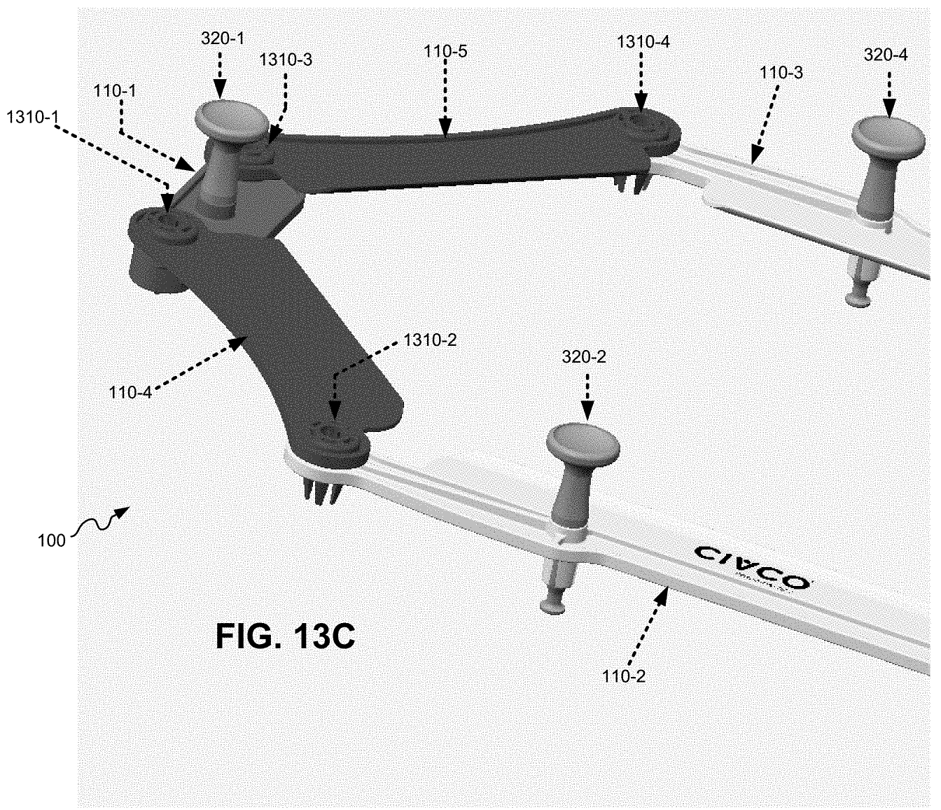

FIGS. 13A, 13B, and 13C depict another exemplary implementation of the multi-profile mask 100 of FIG. 1 in which mask 100 includes five profiles 110. As shown in FIG. 13A, profile 110-1 may be first attached to base 210 using a base fastening mechanism that attaches to base fastening hole 400-1 (not shown). Profiles 110-2 and 110-3 (not shown in FIG. 13A) may further be attached to base attachment holes 400-2 and 400-4 of base 210 using base fastening mechanisms. Referring to FIG. 13B, after attachment of profile 110-1 to base 210, profiles 110-2 and 110-3 may be attached to base via base fastening holes 400-2, 400-3, 400-4 and 400-5 (not shown) using base fastening mechanisms 320-2, 320-3, 320-4 and 320-5. Subsequent to attaching profiles 110-2 and 110-3 to base 210, profile 110-4 may be attached to profiles 110-1 and 110-2 using fastening mechanisms 1300-1 and 1300-2, and profile 110-5 may be attached to profiles 110-1 and 110-3 using fastening mechanisms 1300-3 and 1300-4. Base fastening mechanisms 320-1, 320-2, 320-3, 320-4, and 320-5 each may include a fastening pin that inserts through a pin hole in the profile 110 into a corresponding hole 320 in base 210. Fastening mechanisms 1300-1, 1300-2, 1300-3, and 1300-4 each may include a fastening pin that inserts through a pin hole in the profile 110-4 or 110-5 into a corresponding pin hole in the underlying profile 110 For example, fastening mechanism 1300-1 includes a fastening pin that inserts through a pin hole in profile 110-4 into a corresponding pin hole in profile 110-1, fastening mechanism 1300-2 includes a fastening pin that inserts through a pin hole in profile 110-4 into a corresponding pin hole in profile 110-2, fastening mechanism 1300-3 includes a fastening pin that inserts through a pin hole in profile 110-5 into a corresponding pin hole in profile 110-1, and fastening mechanism 1300-4 includes a fastening pin that inserts through a pin hole in profile 110-5 into a corresponding pin hole in profile 110-3.

FIG. 13C depicts a variation of the implementation of FIG. 13B, in which fastening mechanisms 1300-1, 1300-2, 1300-3 and 1300-4, that include fastening pins in FIG. 13B, have been replaced with fastening mechanisms 1310-1, 1310-2, 1310-3, and 1310-4 that instead each include a profile fastening mechanism and a fastening hole that act together as a snap fit joint. For example, fastening mechanism 1310-1 includes a first fastening hole, disposed on profile 110-1 (left side of profile 110-1 in FIG. 13C) that mates with a first profile fastening mechanism, disposed on profile 110-4 (left side of profile 110-4) that further includes multiple beam catches (e.g., barbed beam catches) that act in conjunction as a snap fit joint for fastening profile 110-4 to profile 110-1. The multiple beam catches of the first profile fastening mechanism are directed downwards, as shown in FIG. 13C, such that they mate with the first fastening hole in profile 110-1 when profile 110-4 is attached to profile 110-1. Additionally, fastening mechanism 1310-2 includes a second fastening hole, disposed on profile 110-2 (left side of profile 110-2 in FIG. 13C) that mates with a second profile fastening mechanism, disposed on profile 110-4 (right side of profile 110-4) that further includes multiple beam catches that act in conjunction as a snap fit joint for fastening profile 110-4 to profile 110-2. The multiple beam catches of the second profile fastening mechanism are directed downwards, as shown in FIG. 13C, such that they mate with the second fastening hole in profile 110-2 when profile 110-4 is attached to profile 110-2.

Furthermore, fastening mechanism 1310-3 includes a third fastening hole, disposed on profile 110-1 (right side of profile 110-1 in FIG. 13C) that mates with a third profile fastening mechanism, disposed on profile 110-5 (left side of profile 110-5) that further includes multiple beam catches that act in conjunction as a snap fit joint for fastening profile 110-5 to profile 110-1. The multiple beam catches of the third profile fastening mechanism are directed downwards, as shown in FIG. 13C, such that they mate with the third fastening hole in profile 110-1 when profile 110-5 is attached to profile 110-1. Also, fastening mechanism 1310-4 includes a fourth fastening hole, disposed on profile 110-3 (left side of profile 110-3 in FIG. 13C) that mates with a fourth profile fastening mechanism, disposed on profile 110-5 (right side of profile 110-5) that further includes multiple beam catches that act in conjunction as a snap fit joint for fastening profile 110-5 to profile 110-3. The multiple beam catches of the fourth profile fastening mechanism are directed downwards, as shown in FIG. 13C, such that they mate with the fourth fastening hole in profile 110-3 when profile 110-5 is attached to profile 110-3.

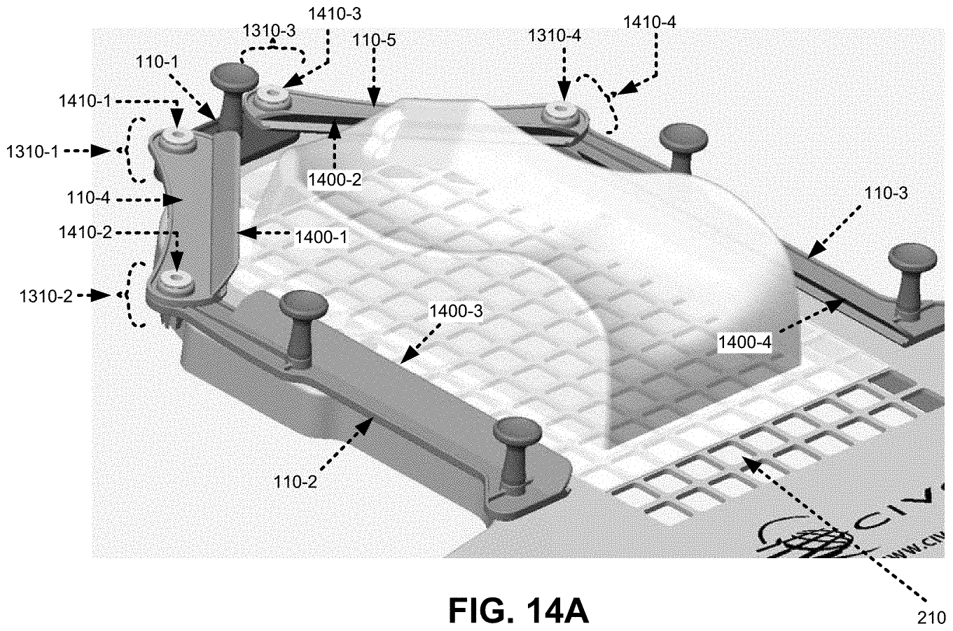

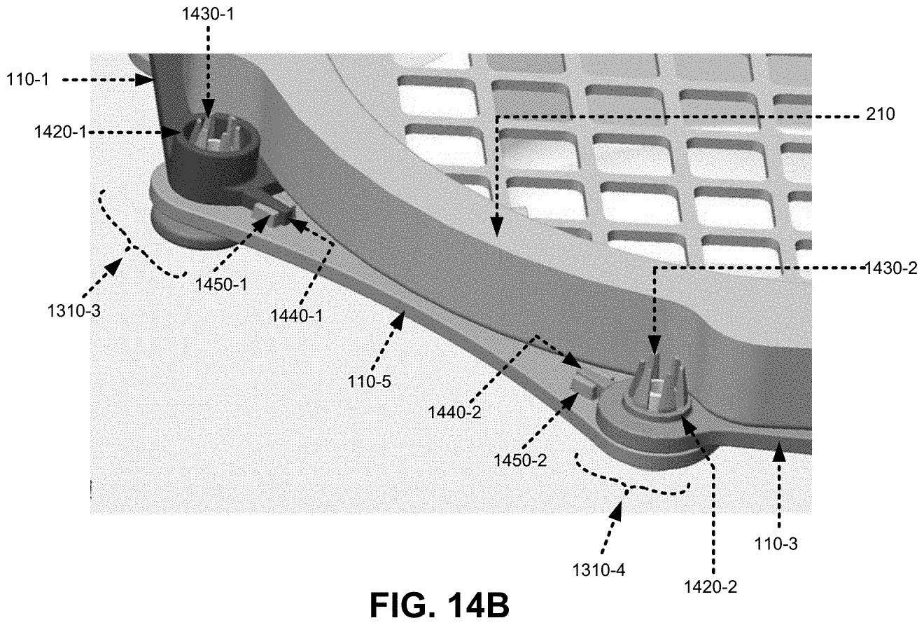

FIGS. 14A and 14B depict a variation of the implementation of FIG. 13C in which angled brackets have been added to profiles 110-2, 110-3, 110-4, and 110-5 for attaching to the sheet 120 of thermoplastic material. As shown in FIG. 14A, profiles 110-2, 110-3, 110-4, and 110-5 have a similar physical configuration to the profiles of the implementation depicted in FIG. 13C, with the addition of a first angled bracket 1400-1 to profile 110-4, a second angled bracket 1400-2 to profile 110-5, a third angled bracket 1400-3 to profile 110-2, and a fourth angled bracket 1400-4 to profile 110-3. Each angled bracket 1400 may connect to an upper surface of a respective profile 110, and extend upwards at an angle to the upper surface of the profile 110. In one implementation, the angle may be a 45 degree angle. Other angles may, however, by alternatively used. The appropriate edge of the sheet 120 of thermoplastic material (not shown in FIGS. 14A and 14B) may be inserted within each bracket 1400 and a bracket fastening mechanism (not shown) may be used to attach each edge of the sheet 120 to each bracket 1400. Various different types of bracket fastening mechanisms may be used that hold the edge of the sheet 120 within, and attach the edge to, a respective bracket 1400.

Additionally, the variation of the implementation of FIG. 13C, depicted in FIG. 14A, includes a filler plugs as a component of fastening mechanisms 1310-1, 1310-2, 1310-3, and 1310-4. Each of fastening mechanisms 1310-1, 1310-2, 1310-3, and 1310-4, as described above with respect to FIG. 13C, includes a profile fastening mechanism and a fastening hole that act together as a snap fit joint. For example, fastening mechanism 1310-2 includes a fastening hole, disposed on profile 110-2 (left side of profile 110-2 in FIG. 14A) that mates with a first profile fastening mechanism, disposed on profile 110-4 (forward portion of profile 110-4) that further includes multiple beam catches (e.g., barbed beam catches) that act in conjunction as a snap fit joint for fastening profile 110-4 to profile 110-2. The multiple beam catches of the profile fastening mechanism are directed downwards, as shown in FIG. 14A, and a filler plug 1410-2 is inserted into a hole in the middle of the multiple beam catches of the profile fastening mechanism of profile 110-4. Filler plug 1410-2 acts to prevent the beam catches of the profile fastening mechanism from squeezing together and releasing, causing profile 110-4 to detach from profile 110-2. Similarly, fastening mechanism 1310-1 includes a filler plug 1410-1, fastening mechanism 1310-3 includes a filler plug 1410-3, and fastening mechanism 1310-4 includes a filler plug 1410-4, with each of the filler plugs 1410 acting, in conjunction with the beam catches of the respective profile fastening mechanisms, similar to the description above with respect to filler plug 1410-2 of fastening mechanism 1310-2.

FIG. 14B depicts another implementation that includes a variation on the implementations of FIGS. 13A-13C. In the implementation of FIG. 14B, the fastening mechanisms 1310, including the fastening mechanisms 1310-3 and 1310-4 depicted, include fastening holes and beam catches that act as a snap fit joint, and with the addition of stabilizing members for controlling the relative position of the interconnecting profiles 110. As shown, fastening mechanism 1310-3 includes a fastening hole 1420-1 and beam catches 1430-1 that operate in conjunction to create the snap fit joint. Further, as shown, profile 110-1, adjacent to fastening mechanism 1310-3, includes a stabilizing member 1424-1 which extends outwards from fastening hole 1420-1 flush with a bottom surface of profile 110-5. Profile 110-5 further includes a stabilizing brace 1450-1, within which stabilizing member 1440-1 fits, so as to stabilize and control the position of profile 110-1 relative to profile 110-5. Additionally, as shown in FIG. 14B, fastening mechanism 1310-4 includes a fastening hole 1420-2 and beam catches 1430-2 that operate in conjunction to create the snap fit joint. Further, as shown, profile 110-3, adjacent to fastening mechanism 1310-4, includes a stabilizing member 1440-2 which extends outwards from fastening hole 1420-2 flush with a bottom surface of profile 110-5. Profile 110-5 further includes a stabilizing brace 1450-2, within which stabilizing member 1440-2 fits, so as to stabilize and control the position of profile 110-3 relative to profile 110-5.

The use of snap fit joints, in the implementations depicted in FIGS. 12A, 12B, 13C, 14A, and 14B, enables attachment and detachment of the profiles 110 to be performed in a simple, user friendly, reliable manner. Though fastening mechanisms 1210 and 1310 have been described as being implemented as snap fit joints for fastening the profiles 110 to one another, other types of fastening mechanisms may be alternatively used. Furthermore, though base fastening mechanisms have been illustrated and/or described, with respect to FIGS. 12A-12B, 13A-13C, and 14A, as being implemented as fastening pins that insert through a pin hole in the profiles 110, other types of base fastening mechanisms may be alternatively used.

The foregoing description of implementations provides illustration and description, but is not intended to be exhaustive or to limit the invention to the precise form disclosed. Modifications and variations are possible in light of the above teachings or may be acquired from practice of the invention. For example, one exemplary implementation is described herein where mask 100 includes three profiles that connect to one another via profile fastening mechanisms. However, in other implementations, a different number of multiple profiles may be used with mask 100. For example, mask 100 may include only two profiles, or four profiles, etc. As an example in which mask 100 has only two profiles 110, a first profile 110 may encompass a first portion of the physical configuration of mask 100, and a second profile 110 may encompass a remaining portion of the physical configuration, with the two profiles having mating fastening mechanisms that enable the two profiles to fasten to one another (e.g., such as when the two profiles are attached to base 200). In one implementation, for example, mask 100 may have a first profile that encompasses one half of the U-shaped configuration depicted in FIG. 8, and a second profile that encompasses a second half of the U-shaped configuration of FIG. 8. Additionally, in this implementation, the two profiles may attach to one another via a snap fit joint such as is depicted FIGS. 3, 12A, 12B, 13C, or 14A. Furthermore, other types of fastening mechanisms may be used with the implementations described herein, such as tracks or fittings that allow profiles to snap together.

Additionally, embodiments have been described herein as having a single sheet of thermoplastic material 120 that attaches between two or more profiles 110 of a mask 100. However, in other embodiments, a mask 100 may include multiple, separate sections of material 120 that attach to the two or more profiles 110 of the mask. Mask 100, therefore, may include multiple separate sheets of material 120 that attach to, and extend between (or out from), the profiles 110 of the mask to stretch over different body parts, or to stretch over different portions of a same body part, of a patient. In these embodiments, each separate sheet of material 120 may not connect to, or come into contact with, any other sheet of material 120 within the mask 100 but may act to stretch over a separate body part, or a different portion of a same body part.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention. Therefore, the above-mentioned description is to be considered exemplary, rather than limiting, and the true scope of the invention is that defined in the following claims.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

What is claimed is:

1. A structure, comprising:
a frame having a plurality of separate profiles or portions; and
a material attached to each of the plurality of separate profiles or portions of the frame for form fitting to one or more body parts,
wherein a first profile of the plurality of separate profiles or portions includes first fastening mechanisms which enable the first profile to be fastened to a second profile of the plurality of separate profiles such that the first profile and the second profile, when fastened together, create a single frame structure.

2. The structure of claim 1, wherein the material comprises a stretchable material and wherein the first profile and the second profile, when fastened together, create a single continuous frame structure.

3. The structure of claim 1, wherein the plurality of separate profiles or portions, when fastened together, creates the single frame structure having a shape that fits around, or over, the one or more body parts.

4. The structure of claim 1, wherein the plurality of separate profiles or portions each include second fastening mechanisms which enable each of the plurality of separate profiles to be fastened to a base.

5. The structure of claim 4, wherein the second fastening mechanisms include a fastening feature that extends through each of the plurality of separate profiles into the base.

6. The structure of claim 1, wherein the first fastening mechanisms comprise at least one snap fit joint.

7. The structure of claim 1, wherein the plurality of separate profiles comprises three profiles, wherein a first profile of the three profiles includes a first female snap fit joint and a second female snap fit joint, wherein a second profile includes a first male snap fit joint that mates with the first female snap fit joint, and wherein a third profile includes a second male snap fit joint that mates with the second female snap fit joint.

8. The structure of claim 1, wherein the one or more body parts comprises a single body part, and wherein the plurality of separate profiles, when fastened together, create a single continuous frame having a shape that fits around, and encloses, three sides of the single body part.

9. The structure of claim 8, wherein the shape comprises a U-shape, and wherein the U shape fits around the top, left side, and right side of the single body part.

10. The structure of claim 2, wherein the plurality of separate profiles or portions further comprises:
at least one additional profile to which the material is also attached, wherein the at least one additional profile does not fasten together with the first profile or the second profile such that the at least one additional profile comprises at least one second frame structure that is discontinuous with the single continuous frame structure comprising the first profile and the second profile.

11. The structure of claim 1, further comprising:
at least two additional profiles;
a second material attached to each of the at least two additional profiles, wherein the second material does not attach to, or connect with, the material attached to each of the plurality of separate profiles or portions of the frame.

12. A medical treatment or testing mask, comprising:
a first profile having a first fastening mechanism;
a second profile, that is separate from the first profile, having a second fastening mechanism that is fastenable to the first fastening mechanism; and
a first sheet of thermoplastic material attached to the first profile and to the second profile,
wherein, when the second fastening mechanism is fastened to the first fastening mechanism, the first profile and the second profile create a single frame structure.

13. The medical treatment or testing mask of claim 12, further comprising:
a second sheet of thermoplastic material attached to the first profile and to the second profile, wherein the second sheet of thermoplastic material does not contact the first sheet of thermoplastic material,
wherein the first sheet of thermoplastic material fits around or over a first body part of a patient, or around or over a first portion of a body part of the patient, and
wherein the second sheet of thermoplastic material fits around or over a second body part of the patient, or around or over a second portion of the body part of the patient.

14. The medical treatment or testing mask of claim 12, wherein the first profile further includes a third fastening mechanism, and further comprising:
a third profile, that is separate from the first profile and the second profile, having a fourth fastening mechanism that is fastenable to the third fastening mechanism,
wherein the sheet of thermoplastic material is further attached to the third profile, and
wherein, when the fourth fastening mechanism is additionally fastened to the third fastening mechanism, the first profile, the second profile, and the third profile create a single continuous frame structure having a shape that fits around or over one or more body parts of a patient.

15. The medical treatment or testing mask of claim 14, wherein the shape of the single frame comprises a U-shape, and wherein the U-shape fits around the one or more body parts of the patient.

16. The medical treatment or testing mask of claim 12, wherein the first profile further includes a third fastening mechanism that enables the first profile to be fastened to a base, and wherein the second profile further includes a fourth fastening mechanism that enables the second profile to be fastened to the base.

17. The medical treatment or testing mask of claim 16, wherein the third fastening mechanism comprises a first pin that is insertable through the first profile into the base and wherein the fourth fastening mechanism comprises a second pin that is insertable through the second profile into the base.

18. A medical treatment or testing mask, comprising:
a first profile having a first fastening mechanism and a second fastening mechanism;
a second profile, that is separate from the first profile, having a third fastening mechanism that is fastenable to the first fastening mechanism;
a third profile, that is separate from the first profile and the second profile, having a fourth fastening mechanism that is fastenable to the second fastening mechanism;
a sheet of thermoplastic material attached to the first profile, the second profile, and the third profile,
wherein, when the third fastening mechanism is fastened to the first fastening mechanism and the fourth fastening mechanism is fastened to the second fastening mechanism, the first profile, the second profile, and the third profile create a single frame structure having a shape that fits around, or over, one or more body parts.

19. The medical treatment or testing mask of claim 18, wherein the shape of the single frame structure comprises a U-shape, and wherein the U-shape fits around the one or more body parts.

20. The medical treatment or testing mask of claim 18, wherein the third fastening mechanism and the first fastening mechanism comprise a first snap fit joint, and wherein the fourth fastening mechanism and the second fastening mechanism comprise a second snap fit joint.
